(19) United States
(12) Patent Application Publication
(10) Pub. No.: US 2020/0146740 A1
(43) Pub. Date: May 14, 2020

(54) Thermodilatation and Thermocompression to Facilitate Targeted Cluster Tissue Ablation and Immune Response

(71) Applicants: William W. Jow, Holmdel, NJ (US); John Mon, Bethesda, MD (US); Stuart E. Katz, Potomac, MD (US); Alan J. Fenn, Wayland, MA (US)

(72) Inventors: William W. Jow, Holmdel, NJ (US); John Mon, Bethesda, MD (US); Stuart E. Katz, Potomac, MD (US); Alan J. Fenn, Wayland, MA (US)

(21) Appl. No.: 16/186,317

(22) Filed: Nov. 9, 2018

(51) Int. Cl. A61B 18/04; A61B 18/02; A61B 18/18

(52) U.S. Cl. CPC A61B 18/04; A61B 2018/00005; A61B 18/18; A61B 18/02

(57) ABSTRACT

Method for treatment of the body with localized energy application in combination with other treatments to facilitate cluster tissue ablation and increase immune response including a method of treatment of a localized treatment area of a body including injection, IV or otherwise to entrap and/saturate a localized treatment area to be used singularly or in combination with: a high electric conductive material comprising a saline solution, hypertonic saline solution, a solution with metallic compounds, metals, nanoparticles, gel, polymers, high-polarity molecules, peptides, liposomes to treat and cause preferential heating and effect secondary conductive heating from cluster sites to adjacent tissues using external or intracavitary energy applicators.

### 1. FIELD OF THE INVENTION

The present invention generally relates to the apparatus and method for treatment of the body with localized energy application in combination with other treatments to facilitate cluster tissue ablation and increase immune response.

### 2. BACKGROUND OF THE INVENTION

Thermotherapy involves the application of thermal energy to body tissues to achieve elevated temperatures, usually in the range of 42-46° C. Oncologic applications of thermotherapy are usually in conjunction with recognized effective conventional treatments including chemotherapy, radiation therapy, and recently immunotherapy. Thermotherapy as adjunctive treatment has demonstrated efficacy as palliative and occasionally as a curative approach to a wide variety of tumors including melanoma (1, 2), sarcoma (3), breast (4, 5), cervical (6), bladder (7), rectal (8) and prostate cancer (9, 10).

Although the application of higher levels of heat by regional or interstitial approaches can have direct cytotoxic effects on tissues, there are several limitations in the technical delivery of such high levels of energy. The heterogeneous distribution of thermal energy resulting from perfusion irregularities of treated tissues and the non-symmetrical confirmation of many tumors as well as their overall size (>3 cm) makes primary and adjunctive thermotherapy a challenge. Many of these technical challenges can be successfully overcome by employing a delivery system already developed and FDA approved, specifically, Prolieve®. Enhanced efficacy of this already proven approach (>100,000 cases to date) resulting in greater focal delivery of heat energy will be achieved with the local tissue infiltration of saline or hypertonic saline solutions, augmenting microwave absorption.

The combined application of thermotherapy with immunotherapy shows great promise for many tumors which have been refractory to conventional chemotherapy or radiotherapy. Current research has demonstrated that hyperthermia amplifies various aspects of the immune response against cancer while simultaneously decreasing immune suppression and escape by these cancers. Additionally, hyperthermia has been shown to preferentially inhibit the repair of certain damaged cancer cells following these conventional approaches (12).

Recent interest in immunotherapeutic agents including the checkpoint inhibitors has focused on the new strategy of inhibiting the immune suppressing signals between various cancer cells and T lymphocytes, a major effector arm of cell-mediated immunity. The checkpoint inhibitors, acting at multiple sites on effector T-cells can effectively overcome immune suppression demonstrated by several cancers. Current research has shown that hyperthermia can augment the effectiveness of check point inhibitors through a variety of proposed mechanisms (12). These include:
1) Up-regulation of both the so-called homing function of T cells as well as augmented function of various antigen complex presenting cells including Dendritic Cells, Natural Killer cells (NK), and Cytotoxic T lymphocytes (CTLs) (13).
2) Thermotherapy has been shown to increase the migration capacity of these various augmenting cells allowing improved infiltration into primary and distant sites with tumors, pre-tumors and/or benign diseases (14).
3) Hyperthermia has been shown to stimulate the production of various cytokines from peripheral T-cells including IL-2 and IFN-y (15). These cytokines amplify both the quantitative as well as the qualitative aspects of the immune effector cells.
4 Additionally, hyperthermia has been shown to down regulate expression of suppressor PD-L1 on some cancer cell lines, directly assisting the mechanism of action for certain check point inhibitors (16).
5) And lastly, hyperthermia has been shown to increase natural killer cell activity against regulatory T cells, so-called Tregs, a sub population of lymphocytes which negatively modulate various aspects of the immune response (17). Higher levels of these Tregs corresponds with more aggressive cancer phenotypes and likely serve as another mechanism of tumor immune escape.

In summary, adjunctive hyperthermia and focal thermotherapy has already shown great widespread application and efficacy in Oncology. Thermotherapy has the proven advantage of being preferably cytotoxic to cancer cells and at the same time to selectively increase various components of immune surveillance. Thus, this invention using the combination of focused heating will enhance with the newly applied immunotherapeutic agents including the checkpoint inhibitors.

### 3. OBJECTS AND SUMMARY OF THE INVENTION

One object of the invention is to provide a method for localized energy application in combination with other treatments to facilitate cluster tissue ablation and increase immune response.

Another object of the invention is to use thermotherapy in combination with compression, an energy source such as, microwaves or laser, and gene or drug therapy.

Yet another object of the invention is to treat a localized treatment area of a body using injection, IV or otherwise to entrap and/saturate a localized treatment area to be used singularly or in combination with: a high electric conductive material.

Still another object of the invention is to open immune response pathways to enhance immune response pathways to a target treatment area.

Yet another object of the invention is to enhance a pathway to distant sites to enhance functions of immunotherapeutic agents with the addition of focused heat.

Still another object of the invention is to provide a method of delivery of immunotherapeutic agents via either/or combination of direct intra-tumoral, intralesional, transluminal, percutaneous, intra-organ injections, topical application and/or systemic delivery of the above immunotherapeutic agents to enhance immune response and/or to unmask and/or unblock immune pathways to primary and distant sites, both benign and cancerous.

It must be understood that no one embodiment of the present invention need include all the aforementioned objects of the present invention. Rather, a given embodiment may include one or none of the aforementioned objects. Accordingly, these objects are not to be used to limit the scope of the claims of the present invention.

In summary, one embodiment of the invention includes a method of treatment of a localized treatment area of a body including injection, IV or otherwise to entrap and/saturate a localized treatment area to be used singularly or in combination with: a high electric conductive material comprising a saline solution, hypertonic saline solution, a solution with metallic compounds, metals, nanoparticles, gel, polymers, high-polarity molecules, peptides, liposomes to treat and cause preferential heating and effect secondary conductive heating from cluster sites to adjacent tissues using external or intracavitary energy applicators. Another embodiment of the invention includes a method of delivery of immunotherapeutic agents via either/or combination of direct intra-tumoral, intralesional, transluminal, percutaneous, intra-organ injections, topical application and/or systemic delivery of the above immunotherapeutic agents to enhance immune response and/or to unmask and/or unblock immune pathways to primary and distant sites, both benign and cancerous. Yet another embodiment of the invention includes a method of delivery of immunotherapeutic agents via either/or combination of direct intra-tumoral, intralesional, transluminal, percutaneous, intra-organ injections, topical application and/or systemic delivery of the above immunotherapeutic agents to enhance immune response and/or to unmask and/or unblock immune pathways to primary and distant sites, both benign and cancerous.

### 4. DETAILED DESCRIPTION OF THE INVENTION

While the instant invention will be described with respect to a preferred embodiment where the bodily conduit is the urethra and prostatic tissue is to be treated by thermotherapy, the combination of compression, an energy source such as, microwaves or laser, and gene or drug therapy can be used to achieve the above goal in other bodily conduits or intracavity sites including, but not limited to, cardiovascular, esophageal, nasal pharynx, and rectal cavities or organs accessible by body conduits such as lung, liver, ovaries, and etc. That is, it is a goal of the instant invention to open up bodily conduits so that the normal function of that conduit is not hampered and to treat both diseased and/or benign sites, as well as the relief of pain, by delivering applicable gene modifiers, drugs or medication to the targeted area. The power to the energy-emitting source for heat or light, and diameters and shaping of the compression balloon and catheter will vary depending upon the tissue or bodily conduit or organ to be treated and the coated material on the compression balloon.

The use of checkpoint inhibitors has recently been used; however, the full extent of its ability to increase apoptosis and to un-inhibit immune response has been limited. This inventive method to enhance heating to the effective area with heat will enhance the ability of the intent of checkpoint inhibitors thus opening the immune response pathways and synergistically increase and enhance the immune response not otherwise achieved.

The selective irradiation according to the method produces sufficient heat to create DNA damage and it is theorized that the protein, which is responsible for the ability of the cancer cells to repair themselves, is removed or deleted from its association with the DNA molecule during the heat achieved by the energy applicator according to the invention. As a result of the removal of this protein, cancerous cells should die naturally by the apoptosis process. Cytotoxins or substances that poison living cells are associated with radiation, chemotherapy, or heat. It is theorized that these cytotoxins damage the DNA molecule deleting the protein responsible for cell repair. Removal or deletion of the protein responsible for repair will enhance the ability of the cytotoxins to cause apoptosis and necrosis of cancerous cells. The DNA damage caused by heat as described by this invention is a primary adjunctive treatment to improve the intent of checkpoint inhibitors to increase the immune response and to overcome the limitations of current checkpoint inhibitors. Heat damage is known to damage DNA molecules and is not specific to type of tissue, benign, cancerous or otherwise thus is universal and can be used to increase any checkpoint inhibitors to overcome the current specific and limitations of current designed checkpoint inhibitors. The combination of checkpoint inhibitors, focused heat and radiation can be more effective compared to the combination of checkpoint inhibitors and radiation.

While this method may be achieved employing the adaptive microwave phased array technology, focusing energy, in general, may be used to heat and ablate an area of tissue. Heating by endo therapy with the enhanced combination of various sized compression balloons will aid in the retention of the entrapped and saturated material to the localized treatment area. The focused energy may include electromagnetic waves, ultrasound waves or waves at radio frequency. That is, this method applies to any energy that can be focused to heat and ablate an area of tissue to facilitate cluster tissue ablation and immune response.

The rational of using high electric conductive material is further explained below. Microwave energy can be focused to heat a region of tissue. While the focused energy may be the primary heating source, it may be combined with an injection of a substance that increases or enhances heating at the target area. The substance may be saline water or water with or without mixing with a metal such as gold nanoparticles, filaments of stainless steel, or other electrical conducting or heat sensitive substance so that the substance enhances the amount of heat delivered to the target areas which become cluster sites to effect secondary conductive heating to adjacent tissues upon cessation of primary heating energy. Bulk (solid) metals typically cause significant reflection of microwaves and do not heat significantly. Metal powders and metallic compounds will heat rapidly when irradiated with microwaves.

The microwave energy absorption per unit mass (or specific absorption rate, SAR, with units of W/kg) of tissue is directly proportional to both the electrical conductivity σ of the tissue and the square of the magnitude of the applied electric field, denoted |E|, and is inversely proportional to the tissue density (denoted ρ with units of kg/m³):

$$SAR = \frac{1}{2} \frac{σ}{ρ} {\vert E \vert}^{2} \qquad ( 1 )$$

The rise in temperature (ΔT) in a given time interval (Δt) with a given applied SAR is expressed as:

$$Δ T = \frac{1}{c} SAR Δ t \qquad ( 2 )$$

where c is the specific heat capacity of the tissue, and thermal conduction and perfusion effects are ignored. The specific absorption rate (SAR) and rise in temperature defined by Equations (1) and (2), respectively, provide an approximate means to compare the relative heating of tissues and materials with different characteristics.

For example, at 37° C., assuming 915-MHz microwaves, 0.9% physiological saline (9 g/kg) has an ionic conductivity 2.1 S/m. The electrical conductivity of stainless steel is 1.5×10⁶ S/m. The electrical conductivity of gold is 4.1×10⁷ S/m. Fat has an ionic conductivity of about 0.2 S/m.

The density of 0.9% physiological saline is approximately 1 g/mL or 1000 kg/m³. The density of stainless steel is on the order of 8000 kg/m³. The density of gold is on the order of 19,300 kg/m³. The density of fat is 928 kg/m³.

The specific heat of 0.9% physiological saline is approximately 4.2 Jg⁻¹K⁻¹. The specific heat of stainless steel is on the order of 500 J/(kg-K) or 0.5 J/(g-K). The specific heat of gold is on the order of 120 J/(kg-K) or 0.12 J/(g-K). The specific heat fat is approximately 2.5 Jg⁻¹K⁻¹.

Both tissue density and tissue specific heat have an inverse relation in regard to the microwave heating rate of tissue (refer to Equations 1 and 2).

Based on the above parameters, a comparison of the relative heating of physiological saline, stainless steel, gold nanoparticles, and fat is given below.

The relative SAR for a given amplitude of electric field is summarized below for materials and tissue.

$$SAR ( saline ) = \frac{σ}{ρ} = \frac{2.1}{1000} = 0.0021$$

$$SAR ( StainlessSteel ) = \frac{σ}{ρ} = \frac{1 \text{,} 500 \text{,} 000}{8000} = 187.5$$

$$SAR ( Gold ) = \frac{σ}{ρ} = \frac{4 \text{,} 100 \text{,} 000}{193000} = 212$$

$$SAR ( fat ) = \frac{σ}{ρ} = \frac{0.2}{928} = 0.0002$$

The relative temperature rise per given time interval for saline, stainless steel, gold and fat is summarized below.

$$Δ T ( saline ) = \frac{1}{c} SAR ( saline ) = 0.0021 \text{/} 4.2 = 0.0005$$

$$Δ T ( StainlessSteel ) = \frac{1}{c} SAR ( StainlessSteel ) = 187.5 \text{/} 0.5 = 37.5$$

$$Δ T ( Gold ) = \frac{1}{c} SAR ( Gold ) = 212 \text{/} 0.12 = 1767$$

$$Δ T ( fat ) = \frac{1}{c} SAR ( fat ) = 0.0002 \text{/} 2.5 = 0.00008$$

Gold and stainless steel particles or filaments will heat significantly faster than fat by several orders of magnitude. Saline will heat about 6 times faster than fat, while hypertonic saline has an even higher specific heat.

The method may include the steps of inserting an E-field probe sensor to an appropriate depth in the organ tissue (if two or more energy applicators employed), monitoring temperatures of the skin surface adjacent the organ or portion of the body to be treated, positioning at least one energy applicator (i.e., one or more applicators) around the organ or body to be treated, setting the initial power level delivered to each energy applicator, setting the initial relative phase delivered to each energy applicator to focus the energy at the E-field probe positioned in the organ tissue (if two or more energy applicators employed), delivering energy to the at least one energy applicator to selectively irradiate the organ tissue or tissue of the body to be treated with focused energy and treat at least one of cancerous and benign conditions of the organ or body to be treated, adjusting the level of power to be delivered to each energy applicator during treatment based on the monitored skin temperatures, monitoring the energy delivered to the at least one energy applicator, determining total energy delivered to the at least one energy applicator and displaying the total energy in real time during the treatment, and completing the treatment when the desired total energy dose has been delivered by the energy applicators to the organ. The preferred organs to be treated are the breast and prostate and in a preferred method, the energy applicators may be positioned in a ring about the breast (or other organs).

A preferred method for treating cancerous or benign conditions of an organ or body to be treated by selective irradiation of the organ or body tissue with energy may include the steps of injecting a substance that enhances heating to an appropriate depth in the organ tissue or tissue of the body to be treated, monitoring temperatures of the skin surface adjacent the organ or body to be treated, positioning at least one energy applicator about the organ or body to be treated, setting the initial power level delivered to each at least one energy applicator, delivering energy to the at least one energy applicator to selectively irradiate the organ or body tissue with energy and treat at least one of cancerous and benign conditions of the organ or body, adjusting the level of power to be delivered to each at least one energy applicator during treatment based on the monitored skin temperatures, monitoring the energy delivered to the at least one energy applicator, determining total energy delivered to the at least one energy applicator and displaying the total energy in real time during the treatment, and completing the treatment when the desired total energy dose has been delivered by the at least one energy applicator to the organ or body to be treated. That is, the method according to the invention may be achieved with a single applicator and may be any energy that can be focused on the cancerous or benign conditions of the organ or body to be treated.

In another embodiment of the invention, microwave absorbing pads and metallic shielding are attached to microwave thermotherapy applicators and to the breast compression paddles. These safety precautions added to the method reduce the electric-field intensity and temperature outside the primary microwave applicator aperture field in the vicinity of the base of the breast, chest wall region, and head and eyes during adaptive phased array thermotherapy in compressed breast tissue for breast tumor (malignant or benign) treatment. In order to minimize the amount of invasive skin entry points, combined E-field and temperature sensors within a single catheter are used with the method. As a result, only a single minimally invasive skin entry point is required resulting in improved patient comfort and reducing the risk of infection. In an alternate embodiment with a single microwave applicator, an E-field sensor is not required, as temperature monitoring controls the power delivered to the applicator. Thus, it is not necessary to have an invasive skin entry point if surface temperature sensors are employed.

Additionally, adaptive microwave phased array thermo-therapy can be used as a heat-alone treatment for early-stage breast cancer. Alternatively, adaptive microwave phased array thermotherapy can be used in combination with a chemotherapy regimen and/or gene-based modifiers for treatment of the primary breast tumor in locally advanced breast cancer. Alternatively, the breast thermotherapy heat-alone treatment can be used as a pre-surgical tool to reduce the rate of second or third incisions (additional surgery) for lumpectomy patients. An additional use of adaptive micro-wave thermotherapy can be in improved breast cancer prevention in which thermotherapy is used with Tamoxifen or other antiestrogen drug for blocking estrogen from binding to the estrogen receptors of breast carcinomas and for direct cancer cell kill by heat. The preferred heating temperature for heat-alone thermotherapy treatment of targeted tissue and surrounding margins potentially containing microscopic disease is in the range of approximately 43 to 50 degrees Celsius. When combined with check point inhibitors, radiation therapy, chemotherapy, and/or hormonal therapy, the preferred heating temperature of tissue and margins is in the range of approximately 41 to 45 degrees Celsius.

In another method according to the invention, a single air-cooled energy applicator is positioned over a predetermined localized area such as the breast but not limited to the breast to where localized heating is desired. In the case for example of a patient would be used to heat the breast tissue with the temperature of the breast tissue being measured by either an inserted temperature probe or temperature sensors attached to the skin of the breast. This method could be used in cases where the breast does not extend into an aperture formed by two or more energy applicators (in a so-termed small breasted patient), or the tumor or tissue to be treated is located at the edge of the aperture formed by the applicators. Depending upon the position of the tumor or tissue to be treated, the patient may lie in either prone or supine to receive treatment from the single air-cooled energy applicator.

The breast tissue may be compressed toward the chest wall by means of a tubular shaped material or band that encircles the patient's torso region. The width of the material may correspond to the width of the breast being treated so that it flattens the breast thereby reducing blood flow in the vicinity of the tumor or tissue to be treated and reducing the depth of the tumor or tissue to be treated relative to the skin.

In yet another method according to the invention, the single applicator would be positioned over the breast or superficial areas having a benign or cancerous tumor, such as the head, neck, torso, arms or legs so that emitted energy is aimed at one of a tumor (treatment for cancer or benign conditions) and an upper portion of the breast where a majority of breast cancers occur (prevention of cancer).

A non-invasive temperature monitoring system is preferred although an invasive temperature probe may be employed depending upon the location of the treated tissue and ability to achieve the therapeutic temperature at the treated tissue. For example, with a single applicator, one or more surface temperature sensors may be used to monitor the skin temperature and the output of which then would be used as feedback signals to control the microwave power level delivered to a microwave applicator. A microwave energy dose of up to approximately 360 kilojoules, preferably about 90 kilojoules (e.g., 200 Watts of microwave power for about 30 minutes, preferably about 50 Watts of microwave power for about 30 minutes) may be administered to the breast to be treated to destroy a tumor prior to lumpectomy or microscopic breast cancer cells following a lumpectomy, for example.

Certain proteins are known to allow cancer cells to spread, whereas other proteins prevent cancer cells from spreading. In the case of breast cancer, high levels of the anti-apoptotic protein Bcl-2 are found in early-stage breast cancers, particularly those cancer cells that are estrogen receptor (ER) positive and tumor suppressor protein p53 immunonegative.

The Bcl-2 family of proteins reduces programmed cell death (known as apoptosis) in breast cancer cells so that the cancer cells do not die fast enough and subsequently spread (Zapata, et al, “Expression of Multiple Apoptosis-Regulatory Genes in Human Breast Cancer Cell Lines and Primary Tumors”, Breast Cancer Research and Treatment 35 Vol. 47; pages 129-140, 1998). Other anti-apoptotic protein in breast cancer are Bel-Xv, Mcl-1 and BAG-1. It is assumed that pro-apoptotic proteins such as Bax, Bak, and CPP32 that prevent cancer cells from spreading are not affected by the heat treatment. Similar proteins are associated with other types of tumors and Applicants' invention envisions treatments of various kinds of cancer. The use of heat achieved by the at least one energy applicator selectively heats anti-apoptosis proteins in the treated body site or organ thereby promoting and increasing the production of protein inhibitors for the anti-apoptosis proteins at the tumor area, which will suppress the anti-apoptosis proteins and suppress the spread of cancer and other associated conditions or diseases. That is, the heat formed by providing power to the at least one energy applicator kills the anti-apoptosis proteins or causes the production of protein inhibitors targeted at the anti-apoptosis proteins that suppress the growth of cancer and other conditions.

Similarly, this invention is used to damage DNA to modulate immune response and to be combined with checkpoint inhibitors to overcome shortcomings of specific immune gateways so that increased immune response will travel along the intended pathways to cause the prevention and/or treatment to distant and local sites.

The selective irradiation according to the method induces sufficient heat to create DNA damage and it is theorized that the protein, which is responsible for the ability of the cancer cells to repair themselves, is removed or deleted from its association with the DNA molecule during the heat achieved by the one or more energy applicators according to the invention. The DNA damage as described above via the focused heat will modulate and increase the immune response regardless of the type of tissue heated. In addition to the increased immune response the heat will result of the removal of this protein, cancerous cells should die naturally by the apoptosis process. Cytotoxins or substances that poison living cells are associated with radiation, chemotherapy, or heat. It is theorized that these cytotoxins damage the DNA molecule deleting the protein responsible for cell repair. Removal or deletion of the protein responsible for repair will enhance the ability of the cytotoxins to cause apoptosis and necrosis of cancerous cells.

Applicants further envision a method for the entrapment in saturation of the localized tissue to be heated by injecting a material with high electrical conductivity, such as a saline solution into the low conductivity normal body tissue and then emitting microwave radiation or other energy toward the body to enhance and provide preferentially heating to the desired tissue and will be able to spare healthy tissue not intended to be heated. To localize or pinpoint the energy to be absorbed by the localized tissue to be treated, small doses of a material with a higher electrical conductivity than the surrounding tissues can be injected into a preselected area to entrap and saturate tissue of a body so that the energy is preferentially absorbed at the preselected area or areas thereby enhancing the heating of the preselected area. The injection of the higher electrical conductivity material may be done up to about a half hour before the exposure to microwave radiation or other energy.

A high electric conductive material may be a saline solution, hypertonic saline solution, a solution with metallic compounds, metals, nanoparticles, gel, polymers, peptides, high-polarity molecules, liposomes, peptides, antibodies or biological agents such as viruses or vaccines to enhance heating. The injection of the material with a higher electrical conductivity may be used in combination with other drugs or medicaments to enhance heating of the preselected area. Depending upon the area of the body to be treated, the at least one energy applicator may be external to the body or inserted in a natural cavity of the body (e.g., transurethral, transrectal). A microwave shielding blanket or other protective covering may be used to protect the body area from stray energy.

The exposure to microwave radiation or other energy theoretically should cause the DNA damage which will enhance cell kill, apoptosis, and increase desired immune response. As described earlier microwave radiation or other energy can also be combined with radiation which has been described in the literature to be used to increase the effectiveness of checkpoint inhibitors. The microwave focused heat can be applied to a cancerous tumor and surrounding margins using a temperature of 43 to 44 degrees Celsius for a period of 30 to 60 minutes. The elevated temperature in the tumor and margins will increase the local blood flow and increase the oxygenation of the cancerous tumor and margins making radiation therapy more effective due to decreased hypoxia. The addition of heat with this invention will synergistically increase the modulation and production of immune responses as well as opening the immune pathway to local and distant sites.

In order to treat the prostate with thermotherapy, it is necessary to heat a significant portion of the prostate gland while sparing healthy tissues in the prostate as well as the surrounding tissues including the urethral and rectal walls of a patient. The prostate gland encircles the urethra immediately below the bladder. The prostate, which is the most frequently diseased of all internal organs, is the site of a common affliction among older men, benign prostatic hyperplasia (BPH), acute prostatitis, as well as a more serious affliction, cancer. BPH is a nonmalignant, bilateral nodular tumorous expansion of prostate tissue occurring mainly in the transition zone of the prostate. Left untreated, BPH causes obstruction of the urethra that usually results in increased urinary frequency, urgency, incontinence, nocturia and slow or interrupted urinary stream and, in severe cases, damages to the bladder and kidneys.

This invention also applies to prostate cancer and is a means to stabilize or lower serum PSA levels as a means of prostate management for many men under active surveillance for early diagnosed prostate cancer, including men with elevated or rising PSA and negative prostate biopsies. Furthermore, the use of energy emitters in combination with direct entrapment and saturation of the treatment area and/or in combination to enhance immune response with immunotherapeutic agents such as check point inhibitors and to open the immune response gateway to local and distant sites thus prevention, control or treatment of local and metastatic diseases. This treatment of the prostate includes transurethral microwave thermotherapy in which microwave energy is employed to elevate the temperature of intraprostatic tissue above about 45° C., hereby thermally damaging the tumorous prostate tissue. U.S. Pat. Nos. 5,330,518 and 5,843,144 describe methods of ablating prostate tumorous tissue by transurethral thermotherapy, the subject matter of which is incorporated by reference. However, improvements still need to be made in this type of therapy to further maintain or enhance the patency of the urethra after the thermotherapy treatment. In particular, urine flow is not always improved despite ablation of the tumorous tissue causing constriction of the urethra because edema produced by the transurethral thermo-therapy treatment blocks the urethra passage resulting in patients treated by the above methods to be fitted with catheters for several days or weeks after the thermotherapy treatment.

U.S. Pat. Nos. 5,007,437, 5,496,271 and 6,123,083 disclose transurethral catheters with a cooling balloon in addition to the anchoring or Foley balloon and are incorporated by reference herein. However, these patents circulate fluid, which acts as a coolant for removing heat preferentially from the non-prostatic tissue adjacent thereto, through the cooling balloons. The '083 patent further discloses the use of a thermotherapy catheter system taught by U.S. Pat. No. 5,413,588 that employs chilled water between about 12°-15° C. as the coolant. Chilled water significantly cools the urethra adjacent the cooling balloon. Likewise, the '271 patent describes a coolant as the fluid to keep the urethral wall temperatures cool. This chilling of the urethra does not aid in maintaining an opening within the heated urethra after the cooling balloon is removed, and reduces the therapeutic effect in the tissue immediately adjacent the urethral wall.

The method may by applied by administering focused energy to a localized area within the body using either a single energy applicator or multiple microwave applicators and/or compression of the body with a balloon filled with fluid, or other means of tissue compression, with or without creation of a microenvironment using vasoconstrictive agents, in order to enhance the heating of a predetermined localized treatment area such as but not limited to tumors, cancerous or benign, glands, fatty tissue, organs, both superficial and or deep seated. Single and/or multiple energy applicators could be external beam, intracavitary and/or interstitial. As another means for the administering of focused energy the present invention generally relates to a minimally invasive method for administering focused energy, such as adaptive microwave phased array or single applicator external hyperthermia or intracavitary and/or interstitial hyperthermia, to treat ductal and glandular carcinomas and intraductal hyperplasia, as well as benign lesions such as fibroadenomas and cysts within the body. Another means is to entrap and saturate the localized region with a high electric conductive material within the localized tissue. Another means is to enhance and retain the high electric conductive materials within the defined area from leakage with compression. The tissue to be treated may be in either male or female patients and thus, the method according to the invention may treat small to large areas within the patients. In addition, the method according to the invention may be used to treat healthy tissue containing undetected microscopic pathologically altered cells of high-water content to prevent the occurrence of or the recurrence of cancerous, pre-cancerous or benign lesions. In addition to the treatment of prostate and breast organs, the heating apparatus described above can also be used as a means to heat other body organs, localized lesions both benign or cancerous for the enhancement to treat localized pre-determined areas without damaging larger areas where damage to healthy tissue is not intended.

This invention also relates to a method and means to entrap and saturate the localized treatment area to enhance the selective heating causing apoptosis or cell death in addition to increasing the gateway of improved immune activity permeated by immunotherapeutic agents such as check point inhibitors, non-specific immunotherapeutic agents such as cytokines and peptides, antibodies, viruses and vaccines for both the localized treatment area and pathways to distant sites with tumors, pre-tumors and/or benign diseases. In particular, the present invention relates to an improved and safer glands and localized areas to be treated, and the enhancement of apoptosis, cell kill, and means to deliver preferential focal heating to body organs such as prostate, breast, lungs, kidneys, liver, bladder, pancreas, skin, increased immune response otherwise not possible and to enhance the immunotherapeutic agents to enhance T-cell immunity and/or to neutralize the tumor cell's natural inhibitors in order to allow both apoptosis and to enhance an immune response. This invention also relates to the delivery of immunotherapeutic agents such as check point inhibitors, non-specific immunotherapeutic agents such as cytokines and peptides, antibodies, viruses and vaccines to overcome short comings of the current methods of delivery. This invention relates to the direct intra-tumoral, intralesional, intra-organ injections, topical application and/or systemic delivery of the immunotherapeutic agents and/or in combination with systemic injection of the immunotherapeutic agents to synergistically enhance immune response from both internal and external to the tumor. This invention further is to enhance treatment with the ability to heat the localized treatment area with the entrapment and/or saturation of the tumor to enhance selective heating by the various means as described above. Furthermore, the localized heating results in enhanced immune response to further unmask and/or unblock the natural inhibitors; thus, will synergistically increase immune response caused by the heat and also allow the immunotherapeutic agents including the checkpoint inhibitors to better perform as intended. Thus, this methodology is a better means of enhanced delivery of the immunotherapeutic agents, which is a significant improvement over current immunotherapeutic agents which work on a limited basis.

This invention thus relates to the enabling apparatus to localize heating to a method to entrap and saturate the desired heating area via direct injection of saline or hypertonic saline solution or other materials with higher conductivity material, such as metals, directly to the localized and desired treatment area in order to preferentially heat the desired area and lessen the damage to healthy tissues. The invention works via direct injection of saline or hypertonic saline solution or other materials with higher conductivity than body's normal tissues with such material as metals and nanoparticles directly to the localized and desired treatment area to entrap and saturate the localized area to be heated to enhance heating resulting in controlled cell kill, apoptosis, increase the immune response and open the immune response gateway to effect local or distant sites. Thus, the present invention provides an enhanced localized treatment as well as an enhanced systemic treatment.

In addition to a means of providing heat, this invention aids to the entrapment and saturation of the electric conductive material after injection or IV, oral or otherwise once localized to the desired areas to be treated with tissue compression via either balloon or compression paddles, or other means of tissue compression, with or without creation of a microenvironment using vasoconstrictive agents. This invention will minimize the leakage of the material normally expected due to blood flow either normal or increased by heat, thus preferential heating is further achieved while sparing normal tissue otherwise damaged by the heat.

This invention also relates to the enabling apparatus to localize heating to a method to enhance the desired heating area, in order to cause DNA damage and switching on heat-inducible heat shock promoters to stimulate and modulate an immune response. Thus, this invention provides a method to efficiently and effectively cause the increase in immune response to overcome the blockage of immune pathways which restricts the full impact of current immunotherapeutic agents including the checkpoint inhibitors; thus, the addition of controlled local heating enhances the intent and modulation of higher level of immune response. Thus, this invention opens the immune response gateways from the heated region to the immune pathways for other sites within the body, which can prevent, reduce, and treat metastatic growth. Thus, this invention overcomes the limitations of many immunotherapeutic agents including the checkpoint inhibitors acting alone by increasing immune response when combined with radiation when focused heat is added and enhanced by the increased activity and production of heat shock promoters. The combination of immunotherapeutic agents including the checkpoint inhibitors, focused heat and radiation can be administered to patients before or after breast surgery.

Certain proteins are known to allow cancer cells to spread, whereas other proteins prevent cancer cells from spreading. In the case of breast cancer, high levels of the anti-apoptotic protein Bcl-2 are found in early-stage breast cancers, particularly those cancer cells that are estrogen receptor (ER) positive and tumor suppressor protein p53 immunonegative. The Bcl-2 family of proteins reduces programmed cell death (known as apoptosis) in breast cancer cells so that the cancer cells do not die fast enough and subsequently spread (Zapata, et al, “Expression of Multiple Apoptosis-Regulatory Genes in Human Breast Cancer Cell Lines and Primary Tumors”, Breast Cancer Research and Treatment, Vol. 47; pages 129-140, 1998). Other anti-apoptotic proteins in breast cancer are Bcl-X_{L}, Mcl-1, and BAG-1. It is assumed that pro-apoptotic proteins such as Bax, Bak, and CPP32 that prevent cancer cells from spreading are not affected by the heat treatment. Similar proteins are associated with other types of tumors and Applicants' invention envisions treatments of various kinds of cancer. Applicants theorize that the use of heat achieved by the at least one energy applicator, according to the invention, selectively heats anti-apoptosis proteins in the treated body site or organ thereby promoting and increasing the production of protein inhibitors for the anti-apoptosis proteins at the tumor area, which will suppress the anti-apoptosis proteins and suppress the spread of cancer and other associated conditions or diseases. That is, the heat formed by providing power to the at least one energy applicator kills the anti-apoptosis proteins or causes the production of protein inhibitors targeted at the anti-apoptosis proteins that suppress the growth of cancer and other conditions as well as enhancement of immune response as a result of the focused heat.

While this invention has been described as having a preferred design, it is understood that it is capable of further modifications, uses and/or adaptations of the invention following in general the principle of the invention and including such departures from the present disclosure as come within the known or customary practice in the art to which the invention pertains and as maybe applied to the central features hereinbefore set forth, and fall within the scope of the invention and the limits of the appended claims.

We claim:

1. A method of treatment of a localized treatment area of a body including injection, IV or otherwise to entrap and/saturate a localized treatment area to be used singularly or in combination with: a high electric conductive material comprising a saline solution, hypertonic saline solution, a solution with metallic compounds, metals, nanoparticles, gel, polymers, high-polarity molecules, peptides, liposomes to treat and cause preferential heating and effect secondary conductive heating from cluster sites to adjacent tissues using external or intracavitary energy applicators.

2. The method according to claim 1, further comprising the step of using a compression balloon against a bodily conduit to be treated or compression paddles or other means of tissue compression, with or without creation of a microenvironment using vasoconstrictive agents, so that to enhance entrapment of heat or light sensitive material to prevent leakage of material via normal blood flow and/or increased blood flow resulting from heating of the localized treatment area.

3. The method as set forth in claim 1, wherein immune response pathways are opened to enhance immune response pathways to a target treatment area and also a pathway to distant sites to enhance functions of immunotherapeutic agents with the addition of focused heat.

4. The method as set forth in claim 2, wherein the step of compression or decompression physically manipulates the bodily conduit or other organs or localized treatment area with a compression balloon against the bodily conduit to be treated or compression paddles or other means of tissue compression to cause additional DNA damage to enhance immunotherapeutic agents to open immune response pathways, local and distant.

5. The method according to claim 1, wherein additional heat or light is delivered to the compression balloon via one of hot water, radio-frequency, laser, microwave, ultrasound and infrared and/or external single or multiple energy emitting applicators via one of hot water, radio-frequency, laser, microwave, ultrasound and infrared energy.

6. The method according to claim 1, further causing control, prevention, and/or treatment of primary and distant sites, both benign and cancerous, as a result of enhanced apoptosis and immune response.

7. The method according to claim 4, wherein the ability of immunotherapeutic agents such as check point inhibitors, and non-specific immunotherapeutic agents such as cytokines and peptides, antibodies, and biological agents such as viruses and vaccines is enhanced to cause apoptosis and increase immune response with the addition of focused heat.

8. The method as set forth in claim 7, wherein immune response pathways are opened to enhance immune response pathways to a target treatment area and also a pathway to distant sites to enhance functions of immunotherapeutic agents with the addition of focused heat.

9. The method according to claim 1, further causing enhancement of synergistic DNA damage with the combination of focused heat with ionizing radiation to further enhance the ability of the above immunotherapeutic agents to enhance opening pathways of both local and distant sites to enhance apoptosis and immune response.

10. A method of delivery of immunotherapeutic agents via either/or combination of direct intra-tumoral, intralesional, transluminal, percutaneous, intra-organ injections, topical application and/or systemic delivery of the above immunotherapeutic agents to enhance immune response and/or to unmask and/or unblock immune pathways to primary and distant sites, both benign and cancerous. 11. The method according to claim 7, wherein additional heat or light is delivered to a compression balloon via one of hot water, radio-frequency, laser, microwave, ultrasound and infrared and/or external single or multiple energy emitting applicators via one of hot water, radio-frequency, laser, microwave, ultrasound and infrared energy.

12. The method according to claim 7, further causing control, prevention, and/or treatment of primary and distant sites, both benign and cancerous, as a result of enhanced apoptosis and immune response.

13. The method according to claim 7 to further enhance to cause synergistic DNA damage with the combination of focused heat with ionizing radiation to further enhance the ability of the above immunotherapeutic agents to enhance opening pathways of both local and distant sites to enhance apoptosis and immune response.

14. A method of delivery of immunotherapeutic agents via either/or combination of direct intra-tumoral, intralesional, transluminal, percutaneous, intra-organ injections, topical application and/or systemic delivery of the immunotherapeutic agents to enhance immune response and/or to unmask and/or unblock the immune pathways to the primary and distant sites, both benign and cancerous.

15. The method as set forth in claim 4, wherein the addition of focused microwave hyperthermia to one or more modalities of radiation therapy, chemotherapy, and immunotherapy in the treatment of cancers such as the intact breast or the prostate can be used as a pre-surgical and/or a post-surgical treatment method.

16. The method as set forth in claim 4, wherein the addition of focused microwave hyperthermia to one or more modalities of radiation therapy, chemotherapy, and immunotherapy can be used in the treatment of benign conditions such as acute/chronic infectious/non-infectious, metabolic and inflammatory conditions including pain, arthritis, psoriasis, prostatitis, and degenerative diseases.
